(19) United States
(12) Patent Application Publication
(10) Pub. No.: US 2009/0326435 A1
(43) Pub. Date: Dec. 31, 2009

(54) Systems and methods for treating superficial venous malformations like varicose or spider veins

(75) Inventors: Killian O'Dowd, Co Dublin (IE); Edward G. Mackay, II, Largo, FL (US); Aidan Mulloy, Co Dublin (IE)

Correspondence Address: RYAN KROMHOLZ & MANION, S.C., POST OFFICE BOX 26618, MILWAUKEE, WI, 53226, US

(73) Assignee: Green Medical, Inc.

(21) Appl. No.: 12/378,378

(22) Filed: Feb. 13, 2009

Related U.S. Application Data
(63) Continuation-in-part of application No. 11/799,583, filed on May 2, 2007.
Application No. 11/799,583 is a continuation-in-part of application No. 11/446,800, filed on Jun. 5, 2006, now Pat. No. 7,465,312.
(60) Provisional application No. 60/796,656, filed on May 2, 2006.

(51) Int. Cl. A61M 37/00

(52) U.S. Cl. 604/20

(57) ABSTRACT

Systems and methods treat superficial venous malformations, such as varicose or spider veins. The systems and methods distribute a reactive agent, e.g., a light-reactive agent such as talaporfin sodium or verteporfin, at or near an inner wall of a superficial feeder vein leading to the varicose or spider veins. The systems and methods activate the reactive agent by applying energy, e.g. non-thermal light energy at a wavelength that activates the reactive agent to cause localized injury to the inner wall of the superficial feeder vein to close the feeder vein to interrupt blood flow to the varicose or spider veins, which consequently shrink.

### RELATED APPLICATION

This application is a continuation-in-part of co-pending U.S. patent application Ser. No. 11/799,583, filed May 2, 2007 and entitled “Systems and Methods for Treating Superficial Venous Malformations Like Spider Veins,” which is a continuation-in-part of Unites States patent application Ser. No. 11/446,800, filed Jun. 5, 2006 and entitled “Systems and Methods for Treating Superficial Venous Malformations Like Spider Veins” (now U.S. Pat. No. 7,465,312), which claims the benefit of U.S. Provisional Patent Application Ser. No. 60/796,656, filed May 2, 2006, and entitled “Systems and Methods for Treating Superficial Venous Malformations Like Spider Veins,” which are all incorporated herein by reference.

### BACKGROUND OF THE INVENTION

As the large group of so-called baby-boomers advances in age, there are increasing demands for effective, non-invasive treatment of vascular diseases or dysfunctions affecting the vascular system. There are also increasing demands for non-invasive cosmetic surgery to repair conditions that have vascular origins.

Venous insufficiency is a very common condition resulting from decreased blood flow from the leg veins up to the heart, with pooling of blood in the veins. Normally, one-way valves in the veins keep blood flowing toward the heart, against the force of gravity. When the valves become weak and do not close properly, they allow blood to flow backward, a condition called reflux. The blood collects in the veins and they enlarge.

Veins that have lost their valve effectiveness, become elongated, rope-like, bulged, and thickened. These enlarged, swollen vessels are known as varicose veins and are a direct result of increased pressure from reflux. Varicose veins are distinguished from reticular veins (blue veins) and telangiectasias (spider veins), which also involve valvular insufficiency, by the size and location of the veins.

A common cause of varicose veins in the legs is reflux in a leg and thigh vein called the great saphenous vein, which leads to visible pooling close to the skin, called varicose veins. The reflux in the great saphenous vein can also lead to reticular veins (blue veins) and telangiectasias (spider veins). Besides cosmetic problems, varicose veins are often painful, especially when standing or walking. They often itch, and scratching them can cause ulcers.

By closing a section of the great saphenous vein leading to the varicose or spider veins, the twisted and varicosed branch veins shrink and improve in appearance. Once the diseased vein is closed, other healthy veins take over to carry blood from the leg, re-establishing normal flow.

Non-surgical treatments for an incompetent saphenous vein include sclerotherapy. Sclerotherapy involves the injection of a solution into the vein that causes the vein walls to swell, stick together, and seal shut. This stops the flow of blood and the vein turns into scar tissue. Microsclerotherapy uses special solutions and injection techniques that can increase the success rate for removal of smaller spider veins. Ultrasound-guided sclerotherapy involves an interventional radiologist passing a thin tube called a catheter into the vein using ultrasound guidance and injecting substance that causes the veins to scar and close, rerouting the blood to healthier veins. The affected vein forms a knot of scar tissue that is absorbed by the body over time.

Sclerotherapy involves tedious, hard to learn injection techniques. It can lead to side effects like stinging or painful cramps where the injection was made, or temporary red raised patches of skin, or skin sores, or bruises. The treated vein can also become inflamed or develop lumps of clotted blood. Applying heat and taking aspirin or antibiotics can relieve inflammation. Lumps of coagulated blood can be drained.

Laser surgery can be used to treat varicose and spider veins in the legs. Laser surgery sends very strong bursts of light onto the vein, which makes the vein slowly fade and disappear. Laser surgery is more appealing to some patients because it does not use needles or incisions. Still, when the laser hits the skin, the patient can feel a heat sensation that can be quite painful. Laser surgery can cause redness or swelling of the skin, and can cause burns and scars. Depending on the severity of the veins, two to five treatments (15 to 20 minutes each) are generally needed to remove veins in the legs. Moreover, for veins larger than 3 mm, laser therapy is not very practical. Furthermore, the capital cost for purchasing trans-dermal lasers can be quite high, making the treatment relatively costly.

Minimally invasive vein ablation treatment can also be used to treat varicose veins. This minimally-invasive treatment is an outpatient procedure performed using imaging guidance. After applying local anesthetic to the vein, the interventional radiologist inserts a thin catheter, about the size of a strand of spaghetti, into the vein and guides it up the great saphenous vein in the thigh. Then laser or radiofrequency energy is applied to the inside of the vein. This heats the vein and seals the vein closed.

There is need for devices, systems, methods, and protocols that provide minimally invasive, cost effective, and patient-friendly surgical and/or cosmetic surgical treatment of superficial venous malformations, such as e.g., in the treatment of varicose or spider veins. There is also a need for devices, systems, methods, and protocols that provide minimally invasive, cost effective, and patient-friendly treatment of diseases or dysfunctions in any region of the body that can be readily accessed by treatment agents carried by blood; e.g., cancers like breast and prostrate cancer; ear, nose, and throat conditions; periodontal disease; and diseases of the eye.

### SUMMARY OF THE INVENTION

The invention provides devices, systems, methods, and protocols that provide minimally invasive, cost effective, and patient-friendly surgical and/or cosmetic surgical treatment of superficial venous malformations, e.g., varicose or spider veins.

The invention also provides devices, systems, methods, and protocols that provide minimally invasive, cost effective, and patient-friendly surgical treatment of diseases or dysfunctions in regions of the body that can be readily accessed by treatment agents carried by blood; e.g., cancers like breast and prostrate cancer; ear, nose, and throat conditions; periodontal disease; and diseases of the eye.

According to one aspect of the invention, the devices, systems and methods distribute a reactive agent at, in, or near an inner wall of a vein. The reactive agent is characterized in that it can be controllably activated by the application of a prescribed form of energy. The devices, systems, and methods activate the reactive agent by applying the prescribed form of energy to activate the reactive agent by use of an intravascular device. The activation of the agent causes localized injury to the inner wall of the vein. The prescribed form of energy can comprise, e.g., electromagnetic radiation, and, more particularly, light energy.

According to another aspect of the invention, the devices, systems, and methods distribute a light-reactive agent at, in, or near an inner wall of a vein. The devices, systems, and methods activate the light-reactive agent by applying light energy using an intravascular device at a wavelength that activates the light-reactive agent to cause localized injury to the inner wall of the vein. The light energy is desirably non-thermal and is generated by a low voltage intravascular photoactivation device, comprising, e.g., one or more light-emitting diodes. In one embodiment, the light-reactive agent comprises LS11 (Talaporfin Sodium) that is administered intravenously. In another embodiment, the light-reactive agent comprises verteporfin that is administered intravenously. Devices, systems, and methods that incorporate this aspect of the invention can treat superficial venous disease, like spider veins.

The devices, systems, and methods improve the quality of patient care. The devices, systems, and methods eliminate side effects such as brusing, burning, and skin discoloration. The devices, systems, and methods do not require tedious, hard to learn injection techniques. They do not require high cost trans-dermal lasers. The devices, systems, and method are usable by a large group of practitioners, such as dermatologists, phlebologists, vascular surgeons, and interventional radiologists.

### BRIEF DESCRIPTION OF THE DRAWINGS

FIGS. 1A and 1B are perspective views of a system of devices for treating a superficial venous disease, such as varicose veins using a light-reactive agent, the agent being suited for intravenous injection, the system including a photoactivation device that is sized and configured for intravascular introduction to the targeted tissue site by advancement into and through a blood vessel leading to the targeted tissue site.

FIGS. 2A, 2B, and 2C show an alternative embodiment of a photoactivation device that can be used with the system shown in FIG. 1A.

FIG. 3 is a perspective view of the system shown in FIG. 1A packaged as a kit, with directions for using the devices to treat a superficial venous disease.

FIGS. 4A, 4B, 4C, 4D, and 4E are enlarged views of the distal end of the photoactivation device shown in FIGS. 1A and 2A, showing arrays of light sources in alternative patterns.

FIG. 5 shows an alternative embodiment of a source of a light-reactive agent usable with the system shown in FIG. 1A, the agent being in tablet or capsule form, for oral ingestion.

FIG. 6 shows an alternative embodiment of a source of a light-reactive agent usable with the system shown in FIG. 1A, the agent being in a band aid form for topical application.

FIG. 7 shows an alternative embodiment of a source of a light-reactive agent usable with the system shown in FIG. 1A, the agent being in cream form for topical application.

FIGS. 8A, 8B, 8c, 8D, 9, 10, 11A, 11B, 12, 13, 14, 15A, 15B, 15C, 16A, 16B, and 17 show a representative method of using a system like that shown in FIG. 1A to treat spider veins.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims.

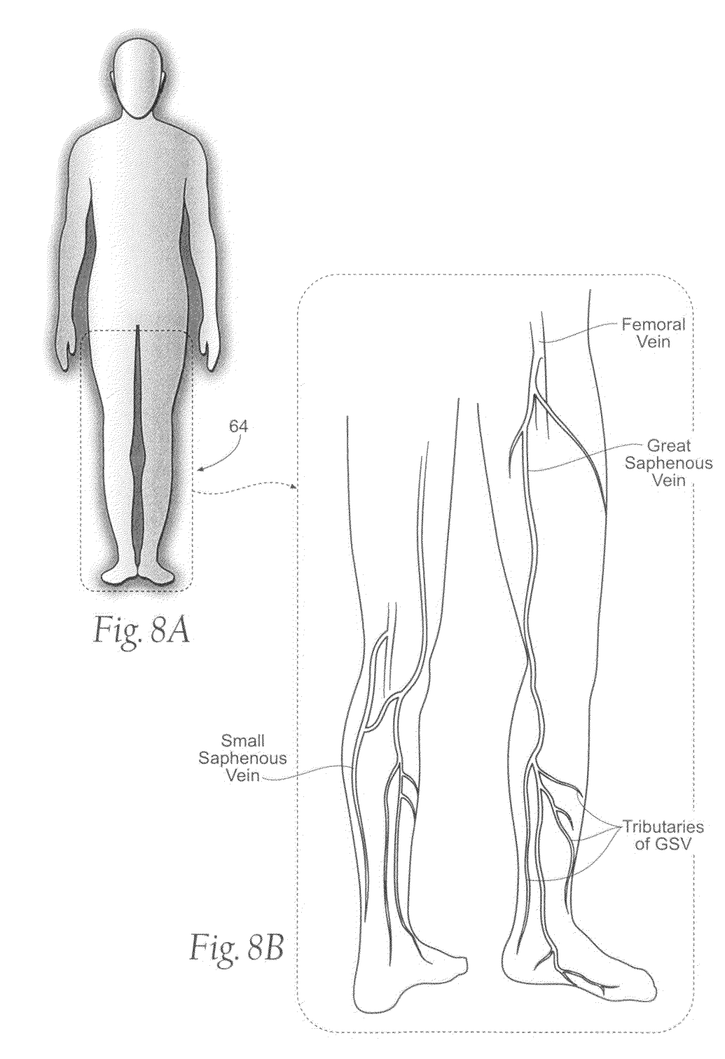

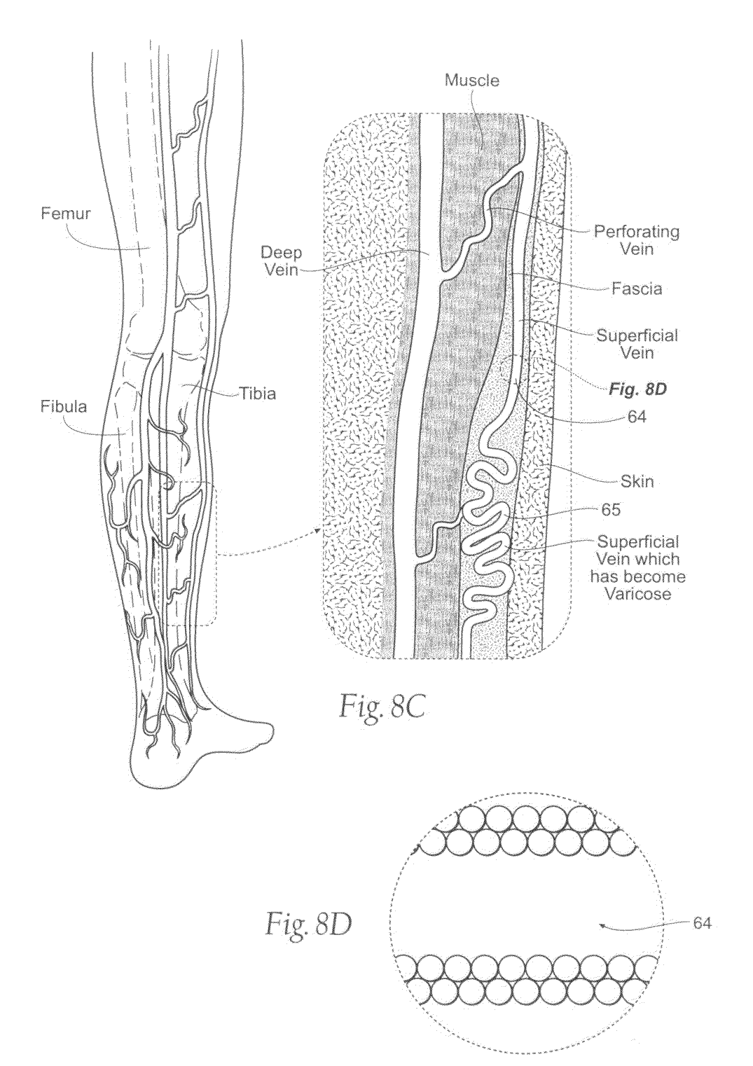

As FIGS. 8B and 8C show, the veins of the legs are divided into two systems: the deep veins (which run deep to the layer of fascia surrounding the muscles) and the superficial veins (which run in the layer of fat just beneath the skin). The superficial veins are visible through the skin (for example, on the foot or around the ankle).

The great saphenous vein (GSV) is the large superficial vein of the leg and thigh. As FIG. 8B best shows, the great saphenous vein is formed from tributaries in the foot, and is visible in many people when they stand, as the vein just in front of the bone on the inner side of the ankle. It runs up the inner side of the calf and the thigh, and at the groin dives to join the main deep vein called the femoral vein. In the thigh it communicates with the femoral vein and receives numerous tributaries. Those from the medial and posterior parts of the thigh frequently unite to form a large accessory saphenous vein which joins the main deep vein at a variable level. As FIG. 8B also shows, the small saphenous vein (SSV) is the other main vein under the skin of the leg, The SSV starts just behind the bone on the outer side of the ankle, and runs up the middle of the back of the calf.

In a number of places in the leg, the superficial saphenous veins and deep veins are linked by perforating veins (or ‘perforators’) (see FIG. 8C). They are called perforators because they perforate the fascial layer surrounding the muscles of the legs.

All leg veins have delicate valves inside them, which normally function to allow the blood to flow only upwards (towards the heart), or from the superficial veins to the deep veins through the perforating veins. The valves protect against the head of pressure that would otherwise exist in the veins of the legs on standing, which would drive blood downward toward the feet. A valve occurs every five to ten centimeters in the main superficial veins of the legs.

Normally, the valves in the perforating veins allow blood to flow only inwards, from the superficial veins to the deep veins. If the valves stop working properly, then blood is pushed out into the superficial veins when the muscles contract. A superficial vein can become varicose because a perforating vein is allowing blood to flow the wrong way (outwards).

In almost any part of the leg, a perforating vein can develop incompetent valves. This allows blood to be pumped outwards under pressure into superficial veins, causing them to become stretched and varicose. The great saphenous vein and its tributaries are the ones that most often form varicose veins. The small saphenous vein and its tributaries can also become varicose, but it is affected much less often than the great saphenous vein.

The systems, methods, and devices disclosed herein are directed to the distribution of a selected reactive agent at, in, or near an inner wall of a vein feeding a varicose or spider vein, such as incompetent segments of a superficial veins such as the great saphenous vein, small saphenous vein, accessory saphenous vein, and perforators. The selected reactive agent is characterized in that it can be reliably and controllably activating in situ by the application of a prescribed form of energy. Once distributed to the targeted site, the reactive agent can be activated in situ by applying the prescribed form of energy. The activation of the reactive agent causes localized injury to the inner wall of the vein. The prescribed form of energy can comprise, e.g., electromagnetic radiation, and, more particularly, electromagnetic radiation in the wavelength spectrum comprising light energy. The devices and system, and their associated methods of use, are particularly well suited for treating superficial venous diseases, such as varicose veins and spider veins.

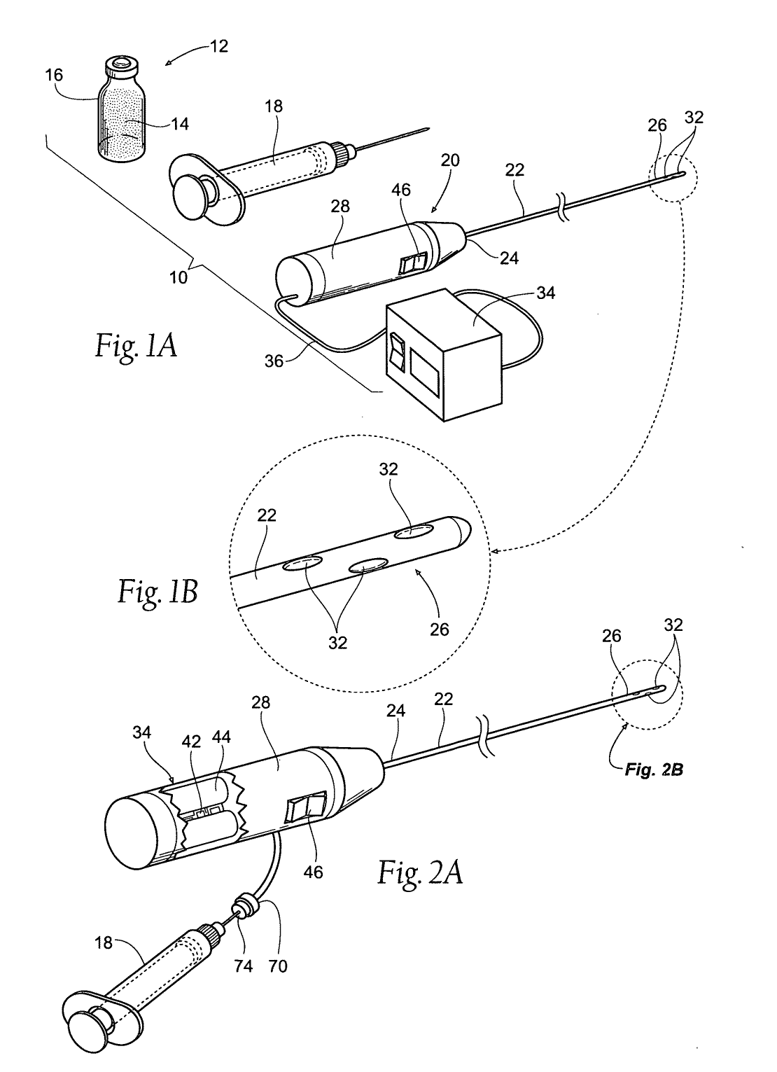

FIG. 1A shows representative devices that together comprise a system 10 for treating a vascular disease or a dysfunction affecting the vascular system using light-reactive agents, i.e., reactive agents that are activated by light energy. The devices and system 10, and their associated methods of use, using light-reactive agents are particularly well suited for treating superficial venous diseases, such as varicose veins or spider veins. For this reason, the devices and system 10, and their associated methods of use will be described in this context.

Still, it should be appreciated that the disclosed devices and system 10, and their associated methods of use are applicable for use in treating other diseases or dysfunctions elsewhere in the body that are not necessarily related to varicose veins or spider veins or their cause, but are nevertheless capable of treatment by light-reactive agents carried by blood. Other conditions that can be treated by light reactive agents using the system 10 or a form of the system 10 include cancer, e.g., breast or prostrate cancer; conditions of the ear, nose, or throat; periodontal disease; and conditions of the eye or sight (opthalmology).

As FIG. 1A shows, the system 10 includes at least one source 12 of a selected light reactive agent 14. The source 12 can be provided in various forms. For example, as shown in FIG. 1A, the source 12 can comprise a conventional vial 16 containing the light reactive agent 14 in solution suited for intravenous injection.

The light reactive agent 14 can comprise any light-reactive drug suited for photodynamic therapy (PDT). PDT is a treatment that uses an agent or drug, also called a photosensitizer or photosensitizing agent, and light energy of a particular selected wavelength. The photosensitizers, which are inert by themselves, bind to proteins found in blood, e.g., lipoproteins. The proteins act as carriers, transporting the photosensitizers to cells targeted for treatment. When exposed to light of the particular wavelength (which varies according to the photosensitizer), the photosensitizer reacts with oxygen. The reaction transforms the oxygen into singlet oxygen and free radicals. The singlet oxygen and free radicals disrupt normal cellular functions and cause cell death.

The light reactive agent 14 can be selected among a group of photosensitizers, depending upon type and location of tissue being treated, as well as the mode contemplated for its introduction into body tissue. Each photosensitizer is activated by light of a specific wavelength. This wavelength determines how far the light can travel into the body. Thus, the physician can select a specific photosensitizer and wavelength(s) of light to treat different areas of the body.

The photosensitizer selected desirably possesses all or some of the following clinically relevant criteria: a commercially available pure chemical; low dark toxicity but strong photocytotoxicity; good selectivity toward target cells; long-wavelength absorbing; rapid removal from the body; and ease of administration through various routes.

Candidate photosensitizers include, but are not limited, to: PHOTOFRIN® (Porfimer sodium—Axcan Pharma, Inc.); FOSCAN® (temoporfin, meta-tetrahydroxyphenylchlorin, mTHPC—Biolitec AG); VISUDYNE® (verteporfin, benzoporphyrin derivative monoacid ring A, BPD-MA—Novartis Pharmaceuticals); LEVULAN® (5-aminolevulinic acid, ALA—DUSA Pharmaceuticals, Inc.); METVIX® (methyl aminolevulinate, MLA or M-ALA—Photocure, ASA); HPPH (2-[1-hexy-loxyethyl]-2-devinyl pyropheophorbide-a, PHOTOCCHLOR—Rosewell Park Cancer Institute); motexafin lutetium (MLu, lutetium(III) texaphyrin, LU-TEX, ANTRIN—Pharmacuclics Inc.); Npe6 (mono-L-aspartyl chlorine e6, taporfin sodium, talaporfin, LS11—Light Science Oncology Inc., Snoqualmie, Wash.); and SnET2 (tin ethyl—etiopurpurin, Sn etiopurpurin, rostaporfin, PHOTREX—Miravant Medical Technologies).

In use, whatever the form, the selected light reactive agent 14 is administered by the system 10 for delivery to a targeted tissue treatment site 64 at, in, or near an inner wall of a vein. In the context of the illustrated embodiment, the targeted tissue site 64 is a sub-dermal region where one or more superficial veins such as the great saphenous vein, small saphenous vein, accessory saphenous vein, and perforators exist that feed or lead to incompetent (varicose or spider) vein segments (this is shown FIG. 8C and will be described in greater detail later).

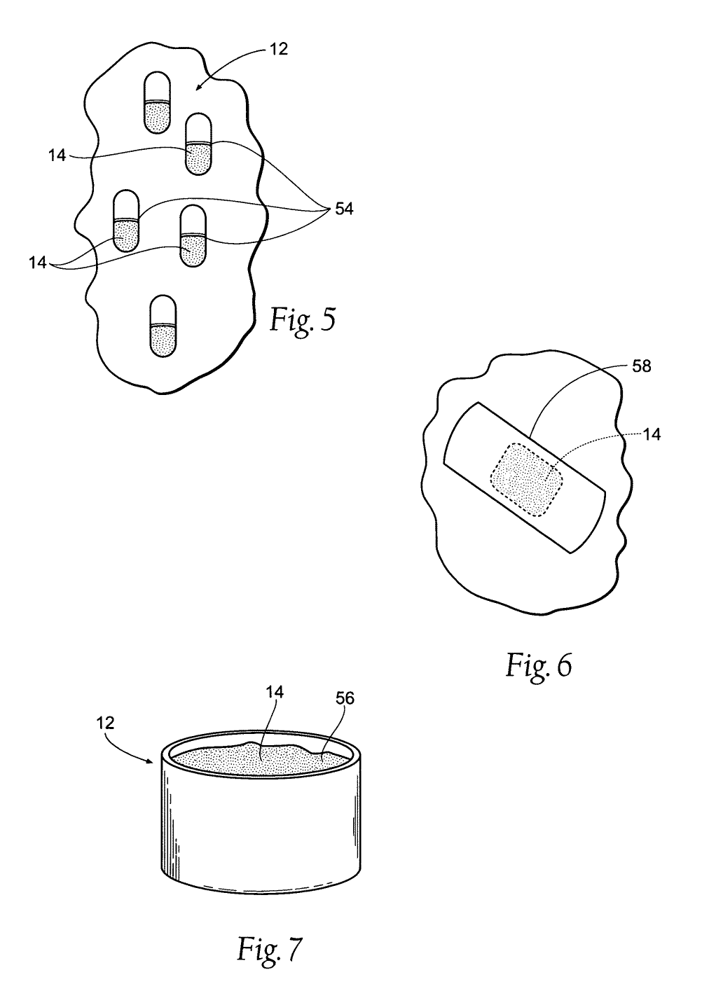

The form for administration will depend upon the form of the source 12. The light reactive agent 14 can be provided in tablet or capsule form 54 (see FIG. 5), which can be ingested orally for absorption by the GI tract for systemic distribution by blood to the targeted tissue treatment site. The tablet or capsule form 54 can incorporate time release features. The tablet or capsule form 54 can also be in the form of an ionosphere to accelerate systemic distribution.

Alternatively, the light reactive agent 14 can be incorporated onto a platform form 58 (see FIG. 6), such as, e.g., a band aid member placed on an exterior skin surface, or as a sub-lingual tab placed on or under the tongue. The light reactive agent 14 can also be applied by pricking the skin.

Alternatively, the light reactive agent 14 can be incorporated into a cream form 56 (see FIG. 7), and the light reactive agent 14 can be applied topically for percutaneous absorption by the skin to the targeted tissue treatment site. The cream form 56 can be applied on exterior skin (e.g., an arm or a leg) or applied within the oral cavity (e.g., by swabbing the gums). The cream form 56 can also incorporate time release features. The cream form 56 can be driven transdermally with the use of ultrasound, or can incorporate dimethyl sulfoxide (DMSO) or aloe cream or similar agent to accelerate transdermal delivery.

It has been discovered that an injectable form of Talaporfin Sodium—available from Light Sciences Oncology, Inc as LS11—can be intravenously administered to effectively treat varicose or spider veins using the system 10 shown in FIG. 1A.

Talaporfin Sodium, together with a special array of light emitting diodes (LEDs), has been tested by Light Sciences Oncology, Inc. in both preclinical and human clinical trials in the United States, Europe and Japan, and has shown efficacy in treating cancer (solid tumors). LS11 material can be activated by shining a LED array at a particular wavelength (664 nm) by a light source into the affected area of tissue.

It has also been discovered that an injectable form of the porphyrin-based photosensitizer called verteporfin—commercially available from QLT, Inc. as VISUDYNE® material (verteporfin for injection)—can be intravenously administered to effectively treat varicose or spider veins using the system 10 shown in FIG. 1. Therefore, FIG. 1A shows the light reactive agent 14 in solution in the vial 16.

VISUDYNE® material has been used, together with a special laser light, to treat abnormal blood vessel formation in the eye, called age-related macular degeneration (AMD) (which, if untreated, can lead to loss of eyesight). VISUDYNE® material can be activated by shining a pre-calculated dose of light at a particular wavelength (689 nm) by a low-energy laser or light source 12 into the affected area of tissue.

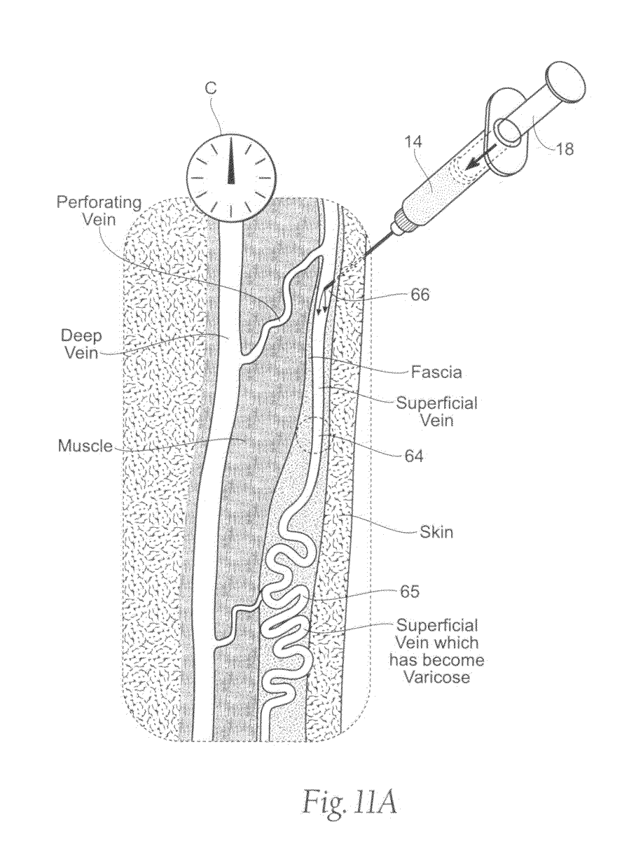

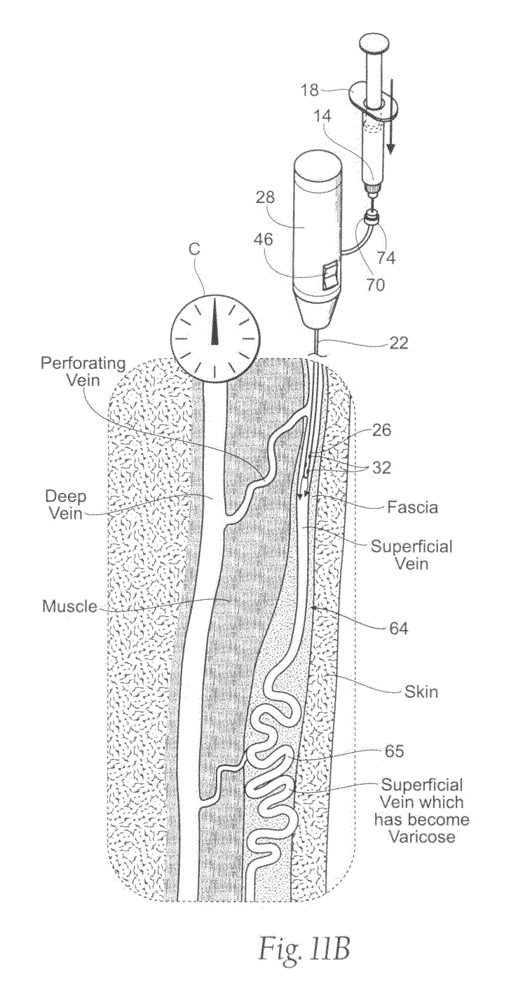

In the context of the illustrated embodiment, where the source 12 comprises an injectable solution of the light reactive agent 14, the device can take the form of a conventional hand-held syringe 18 (as FIG. 1A shows). The syringe 18 draws the light reactive agent 14 in solution from the vial 16 (as shown in FIG. 10) and injects the photodynamic material in solution into the vascular system for transport by the blood flow to the targeted tissue site 64 (as shown in FIG. 11A). The injection site can be locally to tissue in the region to be treated, or directly into a vein or artery serving the region. Instead of a handheld syringe 18, the administration device can take the form of a conventional intravenous (IV) delivery catheter or set coupled to a syringe or other intravenous delivery device or pump.

As FIG. 1A also shows, the system 10 includes a photoactivation device 20 that is sized and configured for intravascular introduction to the targeted tissue site by advancement into and through a blood vessel leading to the targeted tissue site. In this arrangement, the photoactivation device 20 comprises includes an elongated shaft 22 having proximal end region 24 and a distal end region 26.

The proximal end region 24 of the elongated shaft 22 includes a handle 28, as FIG. 1A shows. The handle 28 is sized and configured to be securely held and manipulated by a caregiver outside an intravascular path leading to the targeted treatment site 64 (this is shown in FIG. 11B). By manipulating the handle 28 from outside the intravascular path, the caregiver can advance the elongated shaft 22 through the intravascular path. Image guidance, e.g., CT, radiographic, or another suitable guidance modality, or combinations thereof, can be used to aid the caregiver's manipulation.

The distal end region 26 of the elongated shaft 22 carries at least one or more light sources 32, as FIG. 1B shows. The light sources 32 are also specially sized and configured for manipulation and use within a blood vessel.

The light source or sources 32 have a wavelength or a range of wavelengths. The photoactivation device 20 can include means for controlling the intensity or a range of intensities, spot size or a range of spot sizes, and other operating characteristics of the light source or sources 32 that are conducive to activation the light reactive agent 14 in a desired manner. Desirably, the photoactivation device 20 comprises non-thermal light energy generated by a low-voltage power source (not greater than 12 Volts).

In this arrangement, the system 10 includes a power source 34, as FIG. 1A shows. Under the control of the caregiver or an automated control algorithm, the power source 34 generates energy to illuminate the light source or sources 32. In the embodiment shown in FIG. 1A, a cable 36 plugged into the handle 28 couples the light sources 32 to the power source 34. Supply wires (not shown) passing through a lumen in the elongated shaft 22 from the handle 28 to the light sources 32 convey power to the light sources 32 to illuminate them.

The photoactive device 20 may, alternatively, deliver light through fiber optic cables (e.g., quartz fiber optic cables) and the like through the elongated shaft 22. Alternatively, a fiber optic cable can be inserted through an endoscope or catheter into a targeted internal tissue region (e.g., within a blood vessel or hollow organ) to treat a dysfunction.

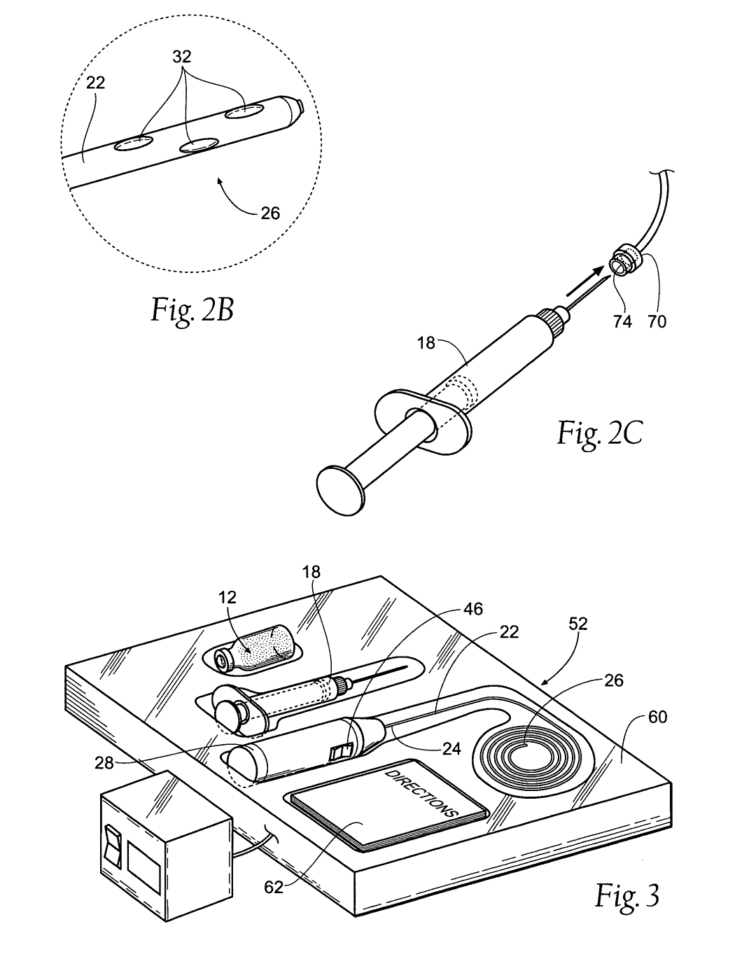

Alternatively, as FIG. 2A shows, the power source 34 can reside within the handle 28. In this arrangement, the handle 28 can encloses a control circuit 42 coupled to a self-contained low voltage (i.e., no more than 12 volts), DC power source 44, such as a battery. The battery 44 can be rechargeable, e.g., by a plug-in connector (not shown), or, alternatively, the battery 44 can be configured to be removed and replaced through a lift-off cover (also not shown) on the handle 28. In this arrangement, the handle 28 includes an on-off switch 46, which activates the control circuit 42.

The light sources 32 can comprise, e.g., lasers, fluorescent, or incandescent lights. The light sources 32 can also comprise light emitting diodes (LED's). LED's can generate high energy light of desired wavelengths and can be assembled in a range of geometry and sizes. The LED's, emitting light in the wave-length(s) that activates the light reactive agent 14. The LED's of a single photoactivation device 20 can be conditioned to deliver multiple wavelengths, so that the photoactivation device 20 can provide a universal platform for different light reactive agents 14. In the illustrated embodiment, where the light reactive agent 14 is LS11, at least one of the wavelengths is 664 nm. Where the light reactive agent 14 is verteporfin, at least one of the wavelengths is 689 nm.

When the reactive agent is activated by another wavelength within the spectrum of electromagnetic energy, e.g., infrared and ultraviolet light, or X-rays and gamma-rays, the source of activating energy comprises a source of the electromagnetic radiation having the other prescribed wavelength.

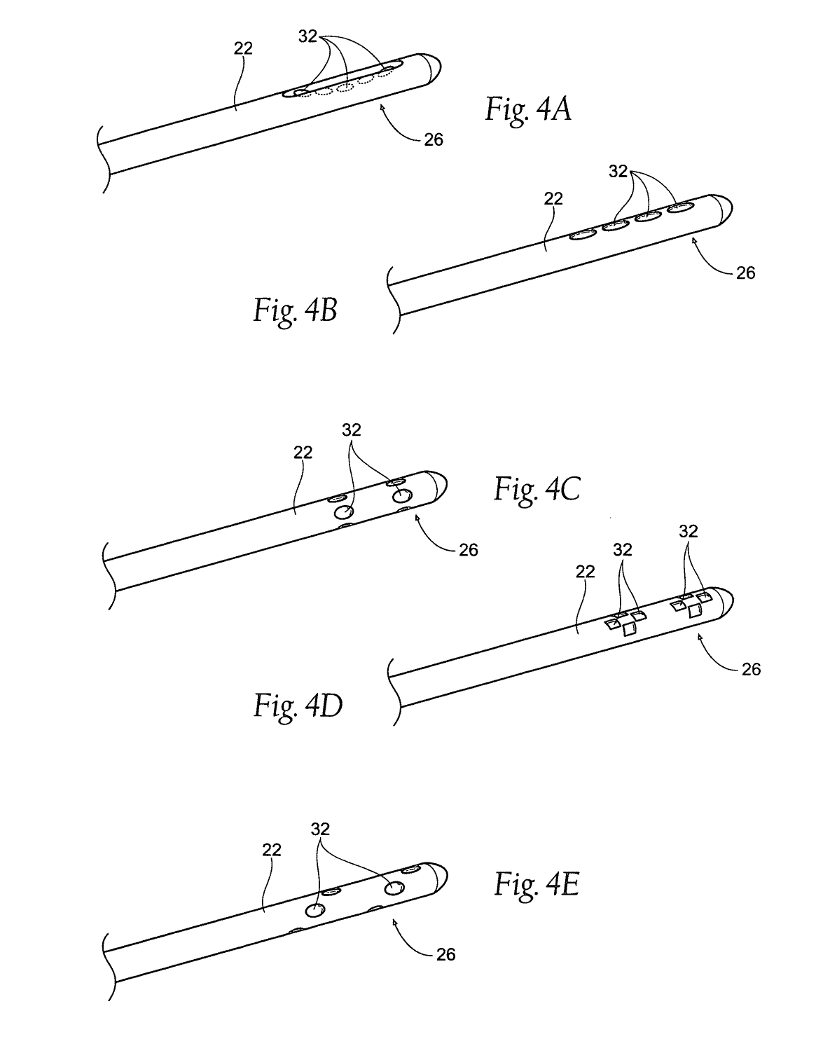

The light sources 32 can be arranged in an array sized and configured to focus at common point, as FIG. 4A shows. Alternatively, as shown in FIG. 4B, the light sources 32 can comprise an array of LED's carried along the distal end region 26 of the elongated shaft, for applying diffused light directly without focusing.

The pattern of light sources 32 can comprise a single linear or curvilinear array (as shown in FIG. 4B); or the pattern of light sources 32 can comprise a square or rectilinear array (as shown in FIG. 4D); or the pattern of light sources 32 can comprise a circle or oval array (as shown in FIG. 4C). The pattern can include linear or curvilinear or zigzag or symmetric or asymmetric arrays of light sources 32 (as shown in FIG. 4E), according to morphology of the targeted tissue region.

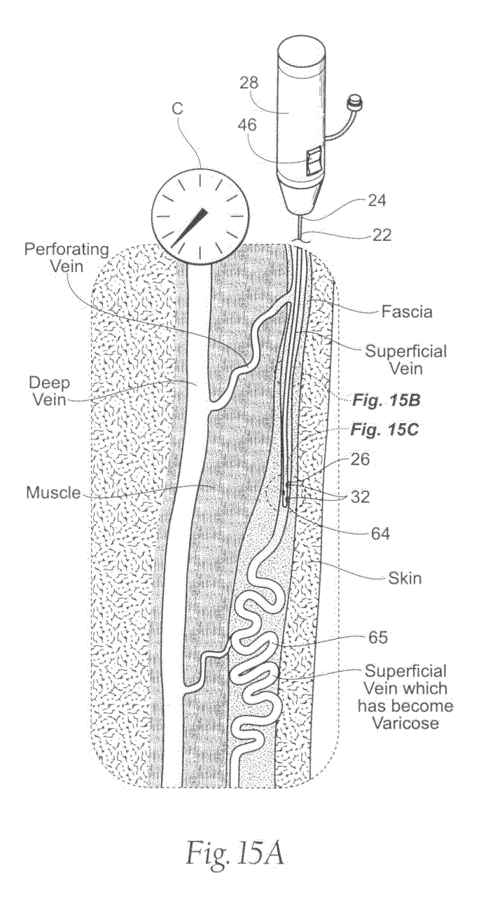

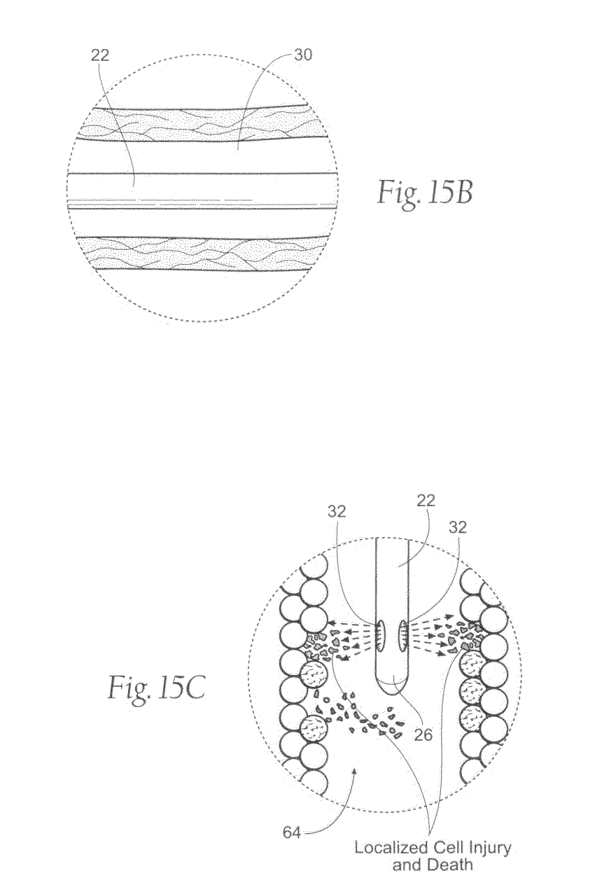

As FIGS. 15A and 15B show, after gaining entrance to the intravascular path, further manipulation of the elongated shaft 22 will advance the distal end region 26 and the one or more light sources 32 situated thereon into proximity and desired alignment with the targeted tissue site 64 along an interior wall of the respective blood vessel where the varicose or spider vein condition exists. Then, light can be applied intravascularly by the light sources 32 to affect the tissue region where the varicose or spider vein condition is located.

The distal end region 26 may include one or more steering wires coupled to a controller on the handle 28. Operating the controller, the caregiver can remotely bend or flex the distal end region 26 within the intravascular path to aid its advancement and desired alignment with the targeted tissue site. The distal end region 26 can include one or more radiopaque markers or bands facilitate visualization and alignment of the light sources 32 with the targeted tissue region within the intravascular path.

As FIG. 3 shows, the various components of the system 10 as just described can be consolidated for use in a functional kit 52. The kit 52 can take various forms. In the illustrated embodiment, the kit 52 comprises a sterile, wrapped assembly including an interior tray 60 made, e.g., from die cut cardboard, plastic sheet, or thermo-formed plastic material, which hold the contents. The kit 52 also preferably includes directions 62 for using the contents of the kit 52 to carry out a desired procedure.

In the illustrated embodiment, every component of the system 10 is contained within the kit 52. Of course, various components can be provided in separate packaging. In this arrangement, the directions 62 still instruct use of the various components separately provided as a system 10.

The directions 62 can, of course vary. The directions may be physically present in the kit 52, but can also be supplied separately. The directions 62 can be embodied in separate instruction manuals, or in video or audio tapes, CD's, and DVD's. The instructions for use can also be available through an internet web page. The directions 62 instruct the practitioner how to use the system 10 to carry out the intended therapeutic treatment. The directions 62 incorporate a method of treatment using the system 10.

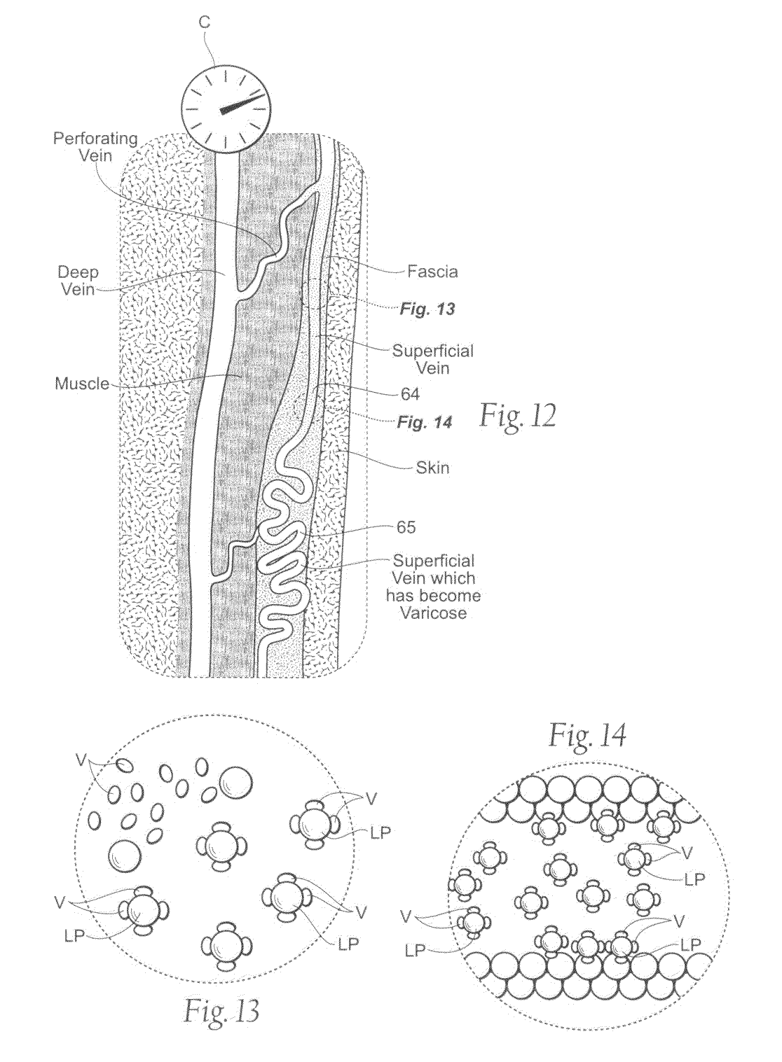

FIGS. 8A, 8B, 8C, 8D, 9, 10, 11A, 11B, 12, 13, 14, 15A, 15B, 15C, 16A, 16B, and 17 show a representative method of using the system 10 shown in FIG. 1A to treat a vascular condition such as varicose or spider veins, which the directions 62 can express in part or in its entirety. As FIGS. 8A, 8B, and 8C show, the method identifies a site where the targeted condition exists, i.e., incompetent segments of a superficial vein(s) where the varicose or spider veins are present. Incompetent segments where varicose spider veins exists are usually easily identifiable by a trained practitioner. Varicose veins are enlarged veins that can be flesh colored, dark purple or blue. They often look like cords and appear twisted and bulging. They are swollen and raised above the surface of the skin. Varicose veins are commonly found on the backs of the calves or on the inside of the leg. Spider veins are similar to varicose veins, but they are smaller. They are often red or blue and close to the surface of the skin. They possess branches or “spider webs” with short jagged lines. Spider veins can be found on the legs and face. They can cover either a very small or very large area of skin.

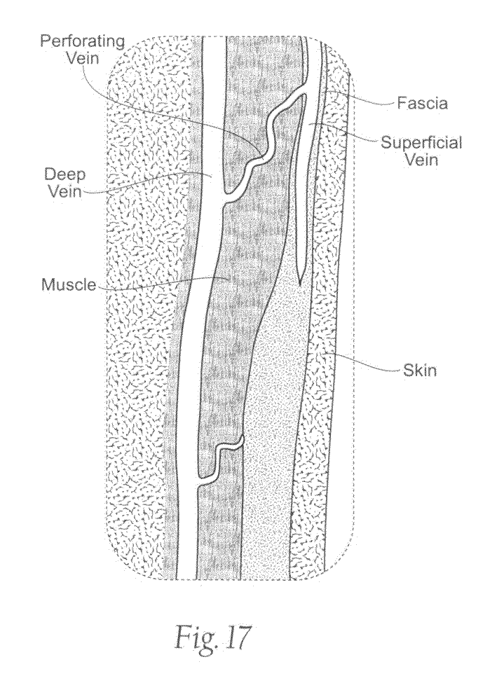

As FIGS. 8C and 8D best show, the method identifies a superficial vein that leads to or “feeds” the incompetent segment. This site will be called the targeted treatment site 64, as shown in FIG. 8C. This can comprise, e.g., a section of the great saphenous vein, the small saphenous vein, the accessory saphenous vein or their tributaries. It is the purpose of the treatment to close the targeted treatment site 64 along the superficial feeder vein, to interrupt blood flow to the incompetent section where the twisted and varicose veins exist. By closing a targeted treatment site 64 within a superficial feeder vein, the twisted and varicosed branch veins close to the skin shrink and improve in appearance or disappear from sight (see FIG. 17). Once the targeted treatment site 64 is closed, other healthy veins take over to carry blood from the leg, re-establishing normal flow.

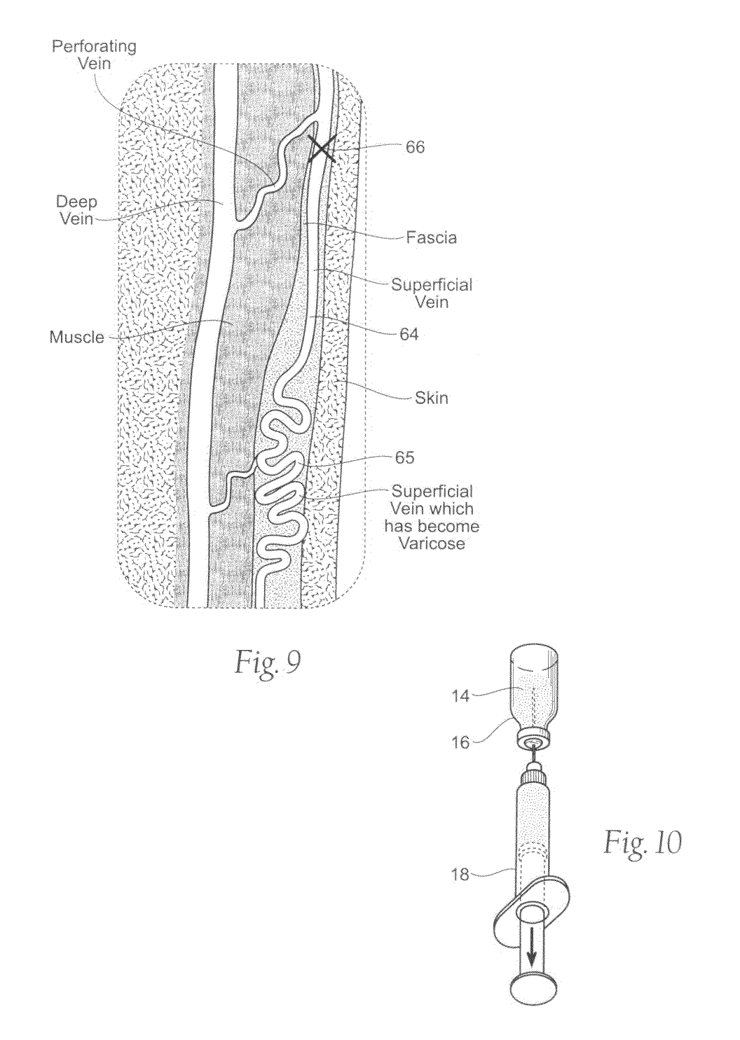

In the illustrated embodiment, the light reactive agent 14 is to be administered intravenously. In this arrangement, an appropriate injection site 66 is identified, as shown in FIG. 9. The injection site 66 is where a selected light reactive agent 14 will administered intravenously by the system 10 (see FIG. 11A) for delivery to the targeted treatment site 64. Desirably, the injection site 66 offers venous access at a distance from the targeted treatment site 64. In this manner, the light reactive agent 14, when injected intravenously, is allowed to become systemic and will be conveyed by venous blood flow to the targeted treatment site 64. FIG. 9 shows, for the purpose of illustration, the injection site being near to or in the same anatomic region of the body (i.e., the leg). However, the injection site 66 need not be adjacent to the treatment site 64, but can be located in an anatomically different region of the body. Venous blood flow in the body will systemically distribute the light reactive agent injected at a remote site throughout the body, including the targeted treatment site 64.

As FIG. 10 shows, the method prepares the light reactive agent 14 for introduction. In the illustrated embodiment, prescribed volume of the light reactive agent 14 is drawn into the syringe 18. The volume to be injected in dependent upon the therapeutic dose that is prescribed, which is, in turn, dependent upon the concentration of the light reactive agent 14 in solution, as well as the morphology of the targeted treatment site 64.

Typically, VISUDYNE® material is commercially reconstituted in saline or glucose solution at desired concentration of about verteporfin 2 mg/mL. At this concentration, a typical dose for a spider vein region can be in the order of 1 cc to 5 cc, but this dosage will of course depend upon the physiology of the individual, including the size and depth of the target treatment site 64, the skin type of the individual, and the body size of the individual. The dosage can be determined by clinical study by physical measurements and titration, or can be selected empirically based upon general anatomic considerations, or a combination of these and other considerations.

As FIG. 11A shows, the method injects the light reactive agent 14 intravenously at the injection site 66. In the illustrated embodiment, the syringe 18 needle injects directly into a superficial vein in the leg. An IV catheter may be used, through which the light reactive agent 14 is injected by syringe or other suitable IV pumping device.

Alternatively (as shown in FIGS. 2A, 2C, and 11B), the elongated shaft 22 of the photoactive device 20 can include an interior infusion lumen terminating with a conventional injection port 70 on the handle 28 and an infusion orifice in the elongated shaft 22, e.g., at the distal end region 26. The injection port 70 carries a septum 74 that can be pierced by the syringe 18 needle, or by a standard male luer fitted to the syringe 18. In this arrangement, the light reactive agent 14 can be injected directly through the photoactive device 20.

The rate of delivery is dependent upon the nature and dosage of the light reactive agent 14 as well as the physiology of the individual being treated. It is desirable to avoid discomfort to the individual, and the rate of delivery selected has this as its primary objective.

It is believed that, given the concentration and volume of the VISUDYNE® material being injected in the illustrated embodiment, an injection period of 20 to 30 seconds is acceptable.

A period of time desirably occurs after injection (as the clocks C in FIGS. 11A/B and 12 indicate), to allow the light reactive agent 14 to become systemic. As FIG. 13 shows, verteporfin V, once injected, attaches to lipoproteins LP in the plasma. The lipoproteins LP carry the verteporin V to the targeted treatment site 64, as FIG. 14 shows. This exposes endothelium of the superficial feeder vein to the verteporin V carried by the lipoproteins LP.

The optimal time period to allow systemic distribution of the light reactive agent 14 in this manner to the targeted treatment site 50 following injection can be determined by clinical study by physical measurements, or can be selected empirically based upon general anatomic considerations, or a combination of these and other considerations.

As the systemic distribution of the light reactive agent 14 occurs, the caretaker can gain access to an intravascular path, e.g., by use of a guide sheath, through which the elongated shaft 22 is passed, or by use of a guide wire, over which the elongated shaft 22 is passed. As FIGS. 15A and 15B show, by manipulation of the elongated shaft 22, the caregiver advances the distal end region 26 and the one or more light sources 32 situated thereon into proximity and desired alignment with the targeted tissue site 64 along an interior wall of the respective superficial feeder vessel where the light reactive agent 14 has been systemically distributed.

As FIG. 15C shows, after allowing a selected time period after injection to pass, the method illuminates the light sources 32 and applies light intravascularly having prescribed characteristics within the vessel where the light reactive agent 14 has systemically distributed. These prescribed characteristics include the wavelength and may also include, but are not necessarily limited to, a desired intensity, a desired spot size, and a desired duration of exposure. The wavelength will depend upon the light reactive agent 14 selected. The intensity, spot size, and duration of exposure of the applied light will depend upon the physiology of the individual being treated and the operating parameters of the system 10, e.g., upon the size of the treatment site 64; the depth of the treatment site 64; the skin type of the individual; the body size of the individual; the distance between the light sources 32 and the targeted tissue site 64; the time of exposure; and the pattern of applying the light. Optimal operating characteristics for the photoactivation device 20 can be determined by clinical study by physical measurements, or can be selected empirically based upon general anatomic considerations, or a combination of these and other considerations. The photoactivation device 20 can apply light either without making direct contact with the skin or by making direct contact with the skin.

As FIG. 15C shows, once verteporfin is activated by light in the presence of oxygen, highly reactive, short-lived singlet oxygen and reactive oxygen radicals are generated. The singlet oxygen and reactive oxygen radicals cause local damage to inner wall or endothelium of the superficial feeder vein. Cells outside of contact with the activated verteporfin, however, are left unaffected.

Treatment by the system 10 and method just described intentionally causes injury to the inner vein walls. By controlling the clinically parameters above described (i.e., the dosage, delivery time and rate, operating conditions of the photoactivation device 20, etc.,) the nature of the injury can be tightly controlled and localized.

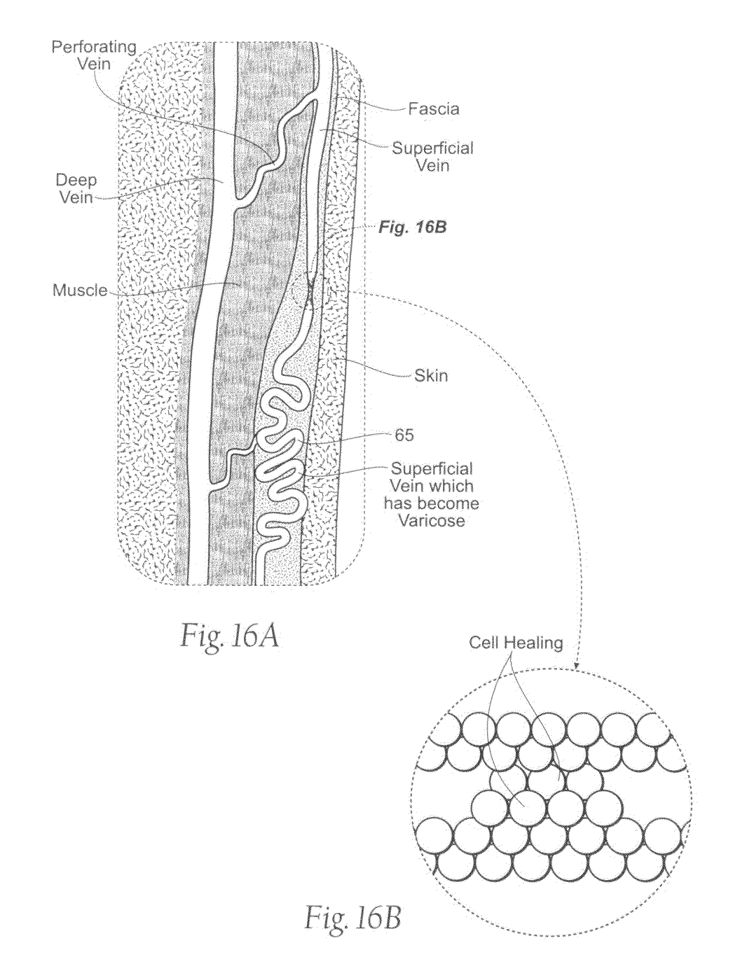

The initial injury to the vein wall evokes a healing process (see FIGS. 16A and 16B). During the healing process, the superficial feeder vein heals shut over time. Blood flow to the section where the twisted and varicose veins exist is interrupted. By closing the superficial feeder vein, the twisted and varicosed branch veins shrink and improve in appearance. Eventually, complete obliteration of the varicose vein condition occurs, as FIG. 17 shows.

It should be appreciated that the devices, systems, methods, and protocols that have been described can provide minimally invasive, cost effective, and patient-friendly treatment of diseases or dysfunctions in all regions of the body that can be readily accessed by treatment agents carried by blood; e.g., cancers like breast and prostrate cancer; ear, nose, and throat conditions; periodontal disease; and diseases of the eye.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

I/We claim:

1. A method for treating an incompetent segment of a superficial vein comprising a varicose or spider vein condition comprising:
identifying a site where an incompetent segment of a superficial vein comprising a varicose or spider vein exists,
identifying as a targeted treatment site a superficial feeder vein leading to the site,
identifying an intravenous injection site offering venous access to the targeted treatment site spaced at a distance spaced from the targeted treatment site,
providing a prescribed volume of a photosensitizing agent in solution that, when exposed to a source of light energy at a selected wavelength, generates singlet oxygen and free radicals without generating heat,
injecting the prescribed volume of the photosensitizing agent in solution at the intravenous injection site,
waiting a prescribed time period to allow the photosensitizing agent in solution to become systemic and be carried by blood proteins into contact with endothelial tissue of an inner wall of the superficial feeder vein at the targeted treatment site,
applying the light energy to the targeted treatment site by a device advanced through an intravenous path into the targeted treatment site, the light energy having a wavelength that activates the photosensitizing agent to generate singlet oxygen and reactive oxygen radicals that disrupt normal cell functions and cause intentional endothelial tissue cell death in the inner wall of the superficial feeder vein at the targeted treatment site and evoke a healing process without affecting non-endothelial tissue cells, and
allowing the healing process to shut and shrink the superficial feeder vein at the targeted treatment site.

2. A method according to claim 1
wherein the photosensitizing agent comprises verteporfin.

3. A method according to claim 1
wherein the photosensitizing agent comprises talaporfin sodium.

4. A method according to claim 1
wherein the light energy comprises light from at least one light emitting diode.

5. A method according to claim 1
wherein the device includes an elongated shaft having a distal end, and at least one light source carried at the distal end.

6. A system for treating an incompetent segment of a superficial vein comprising a varicose or a spider vein condition comprising:
photosensitizing agent in solution that, when exposed to a source of light energy at a selected wavelength, generates singlet oxygen and free radicals without generating heat,
a photoactivation device comprising an elongated shaft having a distal end and at least one light source at the distal end sized and configured to emit light energy at a wavelength that activates photosensitizing agent, and
directions for using the photosensitizing agent in solution and the photoactivation device to treat the varicose or spider vein condition comprising identifying a site where an incompetent segment of a superficial vein comprising a varicose or spider vein exists; indentifying as a targeted treatment site a superficial feeder vein leading, to the site; identifying an intravenous injection site offering venous access to the targeted treatment site spaced at a distance spaced from the targeted treatment site; providing a prescribed volume of a photosensitizing agent in solution that, when exposed to a source of light energy at a selected wavelength, generates singlet oxygen and free radicals without generating heat; injecting the prescribed volume of the photosensitizing agent in solution at the intravenous injection site; waiting a prescribed time period to allow the photosensitizing agent in solution to become systemic and be carried by blood proteins into contact with endothelial tissue of an inner wall of the superficial feeder vein at the targeted treatment site; advancing the photoactivation device through an intravenous path into the targeted treatment site; applying light energy from the at least one light source at the distal end of the elongated shaft to the targeted treatment site, the light energy having a wavelength that activates the photosensitizing agent to generate singlet oxygen and reactive oxygen radicals that disrupt normal cell functions and cause intentional endothelial tissue cell death in the inner wall of the superficial feeder vein at the targeted treatment site and evoke a healing process without affecting non-endothelial tissue cells, and allowing the healing process to shut and shrink the superficial feeder vein at the targeted treatment site.

7. A system according to claim 6
wherein the photosensitizing agent comprises verteporfin.

8. A system according to claim 6
wherein the at least one light source includes at least one light emitting diode.
